(19) United States
(12) Patent Application Publication
(10) Pub. No.: US 2025/0281046 A1
(43) Pub. Date: Sep. 11, 2025

(54) INFRARED THERMOGRAPHY FOR INTRAOPERATIVE FUNCTIONAL MAPPING

(71) Applicant: Northwestern University, Evanston, IL (US)

(72) Inventors: Todd Parrish, Evanston, IL (US); Michael Iorga, Evanston, IL (US); Matt Tate, Evanston, IL (US)

(21) Appl. No.: 19/215,178

(22) Filed: May 21, 2025

Related U.S. Application Data
(62) Division of application No. 17/020,238, filed on Sep. 14, 2020.
(60) Provisional application No. 62/900,063, filed on Sep. 13, 2019.

(51) Int. Cl. A61B 5/00; A61B 5/01; G10L 15/26; G10L 25/78; H04N 23/23

(52) U.S. Cl. CPC A61B 5/0082; A61B 5/015; A61B 5/4064; A61B 5/7405; A61B 5/742; A61B 5/7455; G10L 15/26; G10L 25/78; H04N 23/23

(57) ABSTRACT

A system is described, which provides a realtime, thermal-based intraoperative brain mapping system. In general, the system integrates a thermal camera that provides a map of temperature changes, while simultaneously providing stimuli and recording behavioral responses. The system can implement real-time processing of data to produce functional maps. The present disclosure also provides methods for characterizing the spatial and temporal properties of the thermodynamic response, which can be used to optimize an infrared mapping procedure.

### CROSS-REFERENCE TO RELATED APPLICATIONS

This application is a divisional of U.S. patent application Ser. No. 17/020,238, filed Sep. 14, 2020, which claims the benefit of U.S. Provisional Patent Application 62/900,063, filed on Sep. 13, 2019. Each application is herein incorporated by reference in its entirety.

### BACKGROUND

Functional activation of the cerebral cortex creates a robust increase in local temperature by increasing blood flow and metabolism because of neurovascular coupling. Changes in surface brain temperature while an awake patient performs a motor, sensory, or language task can be used to infer spatial patterns of activity to create functional maps. Awake neurosurgery is used in the management of drug-resistant epilepsy, glioma, and neurovascular malformation, in order to localize seizure and/or physiologic activity. Protection of key functional areas is imperative to avoiding postoperative neurologic deficits.

Currently, direct electrical stimulation (DES) is the most commonly used method of intraoperative surgical mapping, which identifies functionally critical brain regions so they are not resected. However, DES is low spatial resolution (˜1 cm), may provoke seizures, and can only test one area at a time. DES is also limited in that is can evaluate only one region at a time, which limits mapping of functional networks. Clinically, this manifests as false-negatives in inhibitory or regulatory functional regions, such as supplementary motor cortex, leading to postoperative deficits. DES also requires multiple stimulation trials per site, which can take a long time (up to 30 minutes) for complete mapping.

DES is an effective functional mapping tool when a small area is exposed, and when only one or two key functions are considered. However, as the field of glioma is advancing towards more aggressive “supratotal” resections, larger craniotomy areas must be mapped. It is desirable, then, to provide a system for intraoperatively monitoring functional activity that overcomes the drawbacks of DES based techniques.

### SUMMARY OF THE DISCLOSURE

The present disclosure addresses the aforementioned drawbacks by providing an intraoperative thermal imaging system that includes a thermal camera, one or more peripheral devices, and a computer system that includes a processor and a memory. The computer system is configured to receive thermal imaging data from the thermal camera, receive behavioral data from the one or more peripheral devices, and generate a functional map indicative of neuronal activity in a subject using the thermal imaging data and the behavioral data.

It is another aspect of the present disclosure to provide a method for producing a functional map from thermal imaging data. Thermal imaging data are acquired from a patient using a thermal imaging camera while the patient is performing a functional task. The thermal imaging data are processed with a computer system to generate thermal response function (TRF) data indicative of a pattern of temperature change in one or more brain regions of the patient when performing the functional task. A functional map is generated from the thermal response function data using the computer system, wherein the functional map is indicative of neuronal activity in the one or more brain regions in the patient that are associated with performing the functional task.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an example of an intraoperative thermal imaging (“ITI”) system.

FIG. 2 is a flowchart setting forth the steps of an example method for generating functional maps from thermal imaging data.

FIG. 3 is an example of thermal imaging data representative of temperature measurements showing periodic variations associated with respiratory and cardiac cycles.

FIGS. 4A-4C represent example of a functional mapping task protocol.

FIGS. 5A-5C depict example images of functional mapping and related components.

FIG. 6 is a block diagram of an example computer system that can implement methods described in the present disclosure.

### DETAILED DESCRIPTION

Described here are systems and methods for intraoperative functional mapping using thermal imaging. The systems and methods described in the present disclosure enable higher resolution images, faster acquisition speeds, and are non-invasive. The high resolution functional maps obtainable with the systems and methods described in the present disclosure can provide physiologic information, prognostic information, and functional network structures to the neurosurgeon in a time efficient manner.

The intraoperative thermal imaging (“ITI”) techniques described in the present disclosure can be used alternatively or complementary with direct electrode stimulation (“DES”) techniques. For instance, rather than undergoing electrical stimulation to affect task performance, patients may instead perform the same task during thermal imaging data collection. Areas of significant temperature changes (e.g., measured relative to rest) can then be mapped to the performed task. This technique addresses some of the shortcomings of DES. For instance, ITI is high spatial resolution (e.g., about 100 micron) versus DES (e.g., about 1 cm), permitting precise spatial localization. As another example, ITI is non-contact, avoiding potential for stimulation-induced seizures, and therefore maintains the sterile field. As another example, ITI mapping is based on awake tasks and represents a network activation, which allows a more complex mapping beyond a promotion/interruption paradigm. ITI also captures the entire craniotomy window simultaneously, increasing mapping efficiency. As still another example, ITI has high temporal resolution (e.g., 30 Hz), which can capture rapid changes in cortical temperature and has the potential to reveal brain networks. These properties make ITI a natural fit for mapping large craniotomies, such as those in supratotal resection, as DES mapping does not scale well to multiple functional areas.

The systems and methods described in the present disclosure can find use in brain surgery (e.g., tumor, epilepsy, stimulator placement), organ surgery where blood flow is important to monitor (e.g., coronary bypass, transplant), and spinal surgery. In one aspect, the present disclosure provides a real-time, thermal-based intraoperative brain mapping system. In general, the system integrates an infrared (“IR”) thermal camera with devices used to automatically deliver stimuli and record behavioral responses to map motor and language function. The system can implement real-time processing of data, including motion correction, baseline detrending, and statistical analysis of temperature data to produce functional maps. Efficient, automated multi-task protocols can be embedded into this brain mapping system. The infrared recording procedure can be optimized within the surgical workflow, as to maximize signal collection and quality while minimizing treatment interference.

In another aspect, the present disclosure provides methods for characterizing the spatial and temporal properties of the thermodynamic response, which can be used to optimize an infrared mapping procedure. The thermal response function (“TRF”) can be used for measuring the hemodynamic response function (“HRF”), similar to how blood oxygen-level dependent (“BOLD”) is used in functional magnetic resonance imaging (“fMRI”) to monitor oxygenation changes due to the HRF. The spatial and temporal properties of the TRF can be mapped using functional responses, such as well-characterized sensory, motor, and language responses. Through modeling and high resolution (spatial and temporal) IR data, the TRF can be determined across subjects, from which a generalized TRF model can be generated. TRF properties can be leveraged to develop an efficient multi-task mapping protocol. The IR mapping system can be used to generate real-time, high resolution functional maps faster than DES.

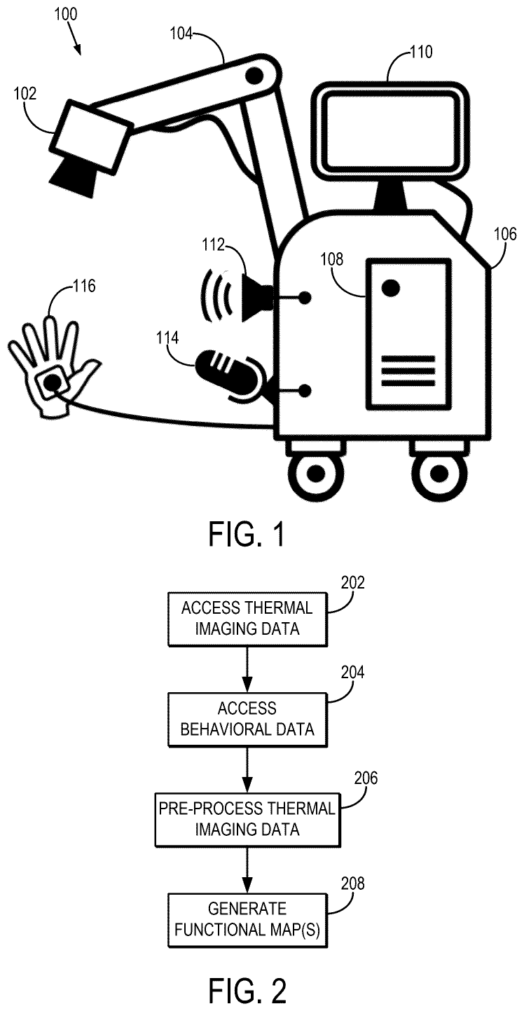

Referring now to FIG. 1, an example of an intraoperative thermal imaging (“ITI”) system 100 according to some embodiments of the present disclosure is shown. The ITI system 100 provides a real-time intraoperative thermal imaging system capable of delivering stimuli and recording the corresponding brain activation and behavioral responses. Real-time algorithms allow for online data monitoring, improving the robustness, efficiency, and reliability of data collection.

The ITI system 100 generally includes a thermal camera 102 that is mounted or otherwise coupled to one end of a moveable support 104. The moveable support 104 is coupled on its other end to a base unit 106. The base unit 106 can include a computer system 108 and one or more peripheral devices, which may include peripheral devices for providing input to the computer system 108 or output from the computer system 108. As an example, the peripheral devices may include a monitor 110 or other visual display, a speaker 112, a microphone 114 or microphone array, and a haptic glove 116, which may be a wired or wireless haptic glove. The base unit 106 is preferably made to be a mobile unit that can be moved as desired. As one example, the base unit can include pneumatic or other caster wheels that enable the base unit 106 to be easily moved as desired. In some configurations, a visual spectrum camera or other imaging device can also be coupled to the base unit 106, such as by way of the moveable support 104 or a second moveable support.

As one non-limiting example, the thermal camera 102 may be an infrared thermal camera, such as a FLIR T1020sc Infrared Thermal Camera (1024×768 resolution, 30 frames/second, 0.02° C. thermal sensitivity) for noncontact measurement of surface brain temperature. In use, the thermal camera 102 can be kept behind a sterile, infrared-transparent barrier, such as a polyethylene barrier.

The moveable support 104 is generally a moveable support that cantilevers the thermal camera 102 over the surgical field. In some instances, the moveable support 104 can be a cantilevered tripod arm mounted to the base unit 106. This type of moveable support 104 permits flexible positioning and orientation of the thermal camera 102 for maximal spatial resolution and ease of use during an intraoperative procedure.

The base unit 106 can be, for example, a stable, mobile cart. The base unit 106 can store equipment, such as the computer system 108 and peripheral devices (e.g., output peripherals, input peripherals).

The computer system 108, which may be stored in the base unit 106, programmed or otherwise configured to control stimulus delivery (e.g., audio, visual, wireless or wired haptic glove), behavioral monitoring (e.g., microphone, wireless or wired haptic glove), real-time data analysis, and communication of mapping results to the neurosurgical team through a custom software interface.

Thermal brain images obtained with the thermal camera 102 can be displayed during data collection, so that data quality can be monitored intraoperatively. The computer system 108 can be programmed or otherwise configured to fuse all of the peripheral device data with a desired temporal resolution, such as a millisecond resolution.

The computer system 108 may implement a user interface, such as a graphical user interface (“GUI”) that enables a user to control all aspects of a thermal mapping process. As one non-limiting example, the user interface can be used to control the delivery of stimuli to a patient by way of one or more output peripheral devices. The output peripheral devices may include speakers, a video or other visual display, and a wireless or wired haptic glove. The haptic glove may include vibrotactile actuators that provide adjustable stimulation to each finger. The haptic glove may also include positional sensors for joint angle measurements. As one example, the haptic glove may be a VMG35 Plus Haptic Glove (Virtual Motion Labs, Dallas, TX).

As a non-limiting example, the wireless haptic glove can be used to sample the position and velocity of the hand and fingers using inertial measurement units. The position-velocity space can be defined as a high-dimensional vector space where each point corresponds to a hand position with a particular velocity. Then, the hand's motion during a task can be described by a path through the position-velocity space. This provides a flexible and precise framework for quantitative task monitoring. For example, to improve task analysis the precise start and end of a movement can be determined from the glove data. Furthermore, the task compliance (e.g., index finger to thumb versus pinky to thumb) can be determined to improve the analysis of the thermal imaging data. Even when the correct task is performed, outliers in performance can be found and eliminated. The task completion matrix will be updated, which may require the subject to repeat a particular task if it was not performed correctly.

For instance, a wireless haptic glove output at a given point in time can be thought of as a vector containing all positional hand information (e.g., joint angles, orientation, pressure). The rate of change for each value can be calculated by subtracting the positional vector from the previous point in time, which yields a hand velocity vector. By concatenating the hand positional and velocity vectors, a complete quantitative description of the hand state at any point in time can be achieved. The hand state space can then be defined as the set of all possible hand state vectors. A typical hand motor task calls for a patient to perform a repetitive sequence of distinct hand movements (e.g., open palm→first→“OK” gesture→open palm . . . ). The sequence of hand state vectors can be thought of as a trajectory in hand state space, with lines connecting all distinct hand positions. Since patients spend most of the task time holding a hand position, the data samples are concentrated around points in state space which correspond to a hand position. These centers can be estimated by applying a k-means algorithm, setting k to the number of distinct hand positions in the task. Each center can then be labeled by popular vote of nearby points, where each point votes according to the intended hand motion when the hand position was collected. This process is entirely subject-specific, and therefore generalizes well to patients with motor disabilities.

Using this construct of hand motion, the automatic task delivery system can perform quality assurance of hand motor mapping tasks in real-time. As one example, if a patient performs an incorrect movement, their hand trajectory will approach the wrong center in state space. As another example, if a patient performs an incomplete movement, the distance to the corresponding center will be large when compared to other samples. In either of these example, the algorithm can repeat the failed task epoch and alert the surgical team to the specific compliance issue. In another example, if a patient is generally noncompliant, k-means label voting will reach a weak majority, or even a plurality. This is unlikely, as patients with severe cognitive, behavioral, or motor limitations are not candidates for awake surgery. Patients can be trained on motor tasks with the haptic glove in the week prior to surgery to improve task compliance. This quality assurance process is computationally efficient and takes fractions of a second to perform. The process rigorously monitors patient compliance and provides specific feedback to the surgical team as needed, with the goal of maximizing data quality while streamlining the testing process.

The quality assurance of task performance is quantitative and can implicitly account for patients with resting hand tremors or dystonias. Combining analysis of behavioral data with automated task administration identifies and repeats erroneous trials to obtain higher data quality for reproducible mapping in an efficient manner.

Behavioral data can be acquired, processed for suitability, and used to inform the analysis of the thermal imaging data or to determine if the stimuli need to be presented again due to lack of patient compliance. During motor/sensory tasks the output of the haptic glove (finger position and velocity, vibratory output) can serve as the stimulus vector. For language tasks, either video or auditory presentation can be used. The subject's vocal response can also be recorded for processing.

During different mapping sessions, it may be desirable to record and interpret the voice of the patient, surgeon, and functional tester. This data can provide timing information for analysis of the thermal imaging data, behavioral data, and other acquired data (e.g., other physiological data that may be recorded, including electrophysiological data). The patient's voice can be isolated using a microphone (which in some instances may be a microphone array) and audio processing techniques, such as spatial filtering.

As an example, when using a microphone array the amplitude of the patient's voice signal will have a specific ratio of amplitudes on each microphone in the array due to the difference in the sound wave path length. By comparing the amplitudes of auditory signals across the microphones, a spatial map of audio sources in the operating room can be constructed. Because the operating room contains numerous audio sources, spatial auditory filtering can be used to isolate the patient's voice from other voices and any background or environmental sounds of noises.

The audio data recorded with the microphone or microphone array can also enable identifying patient cardiac and breathing rate, which can be encoded as beeping from vitals monitoring devices. As one non-limiting example, the microphone array can be constructed by placing multiple (e.g., at least four) omnidirectional microphones in a non-coplanar orientation on the base unit 106 of the ITI system 100. Once the patient's speech is isolated, it can be converted into text using a speech recognition algorithm and displayed on the monitor or other visual display of the ITI system 100. When the patient provides audio responses during a functional task, these responses can be compared to a list of expected responses (e.g., “yes”, “pumpkin”, or “fish”), which can be used in the analysis and determine if the task needs to be repeated.

Using the ITI system 100 described in the present disclosure, it is possible to map functional areas with a spatial resolution of down to 100 μm, which is several orders of magnitude better that the spatial resolution attainable with other techniques, such as direct electrode stimulation (“DES”).

The computer system 108 can further be programmed or otherwise configured to include a suite of mapping software with automated intraoperative task administration and analysis. These software features aid ease of use, which is a primary barrier limiting the adoption of thermography in neurosurgery.

It is contemplated that the ITI systems described in the present disclosure will be advantageous in the neurosurgical environment due to their high precision and ease of use. Temperature is a fundamental biological variable, and its mechanism as a functional contrast is straightforward to understand. Furthermore, infrared thermal cameras are inexpensive. In this way, the ITI systems described in the present disclosure provide a high-performance, but low-cost mapping technology that easily integrates into neurosurgical practice. This will improve patient access to ITI-based mapping beyond high-end academic medical centers, reaching the significant majority of glioma patients who receive care at community hospitals.

Referring now to FIG. 2, a flowchart is illustrated as setting forth the steps of an example method for functional mapping using an intraoperative thermal imaging system. The method includes accessing thermal imaging data with a computer system, as indicated at step 202. The thermal imaging data can be accessed by retrieving previously acquired thermal imaging data from a memory or other data storage device or media. Additionally or alternatively, the thermal imaging data can be accessed by acquiring the thermal imaging data with a thermal camera and communicating or otherwise transferring the data to the computer system (e.g., in real-time).

The method also includes accessing behavioral data with the computer system, as indicated at step 204. The behavioral data can be accessed by retrieving previously acquired data from a memory or other data storage device or media. Additionally or alternatively, the behavioral data can be accessed by acquiring the behavioral data with one or more peripheral devices, such as those described above.

The thermal imaging data can be pre-processed, as indicated at step 206. As one example, the thermal imaging data can be pre-processed to correct for motion. For instance, the brain is a nonrigid organ that is constantly moving due to cardiac (˜1 Hz) and respiratory (˜0.2 Hz) cycles. Even with the high temporal resolution of the thermal camera, the thermal imaging data can be sensitive to small movements because of the high spatial resolution and temperature sensitivity. Motion correction can be implemented using the techniques described below, or any other suitable technique.

After the thermal imaging data have been pre-processed, one or more functional maps are generated from the thermal imaging data, as indicated at step 208. In some implementations, the functional maps are generated from the thermal imaging data using, in part, the behavioral data. For example, functional maps can be generated by computing or otherwise identifying TRFs from the thermal imaging data and using statistical analyses to generate functional maps based on the correspondence between the TRFs, which indicate a coupled neurovascular response to the functional task, and the functional task(s) performed by, or stimuli delivered to, the patient as represented by the behavioral data.

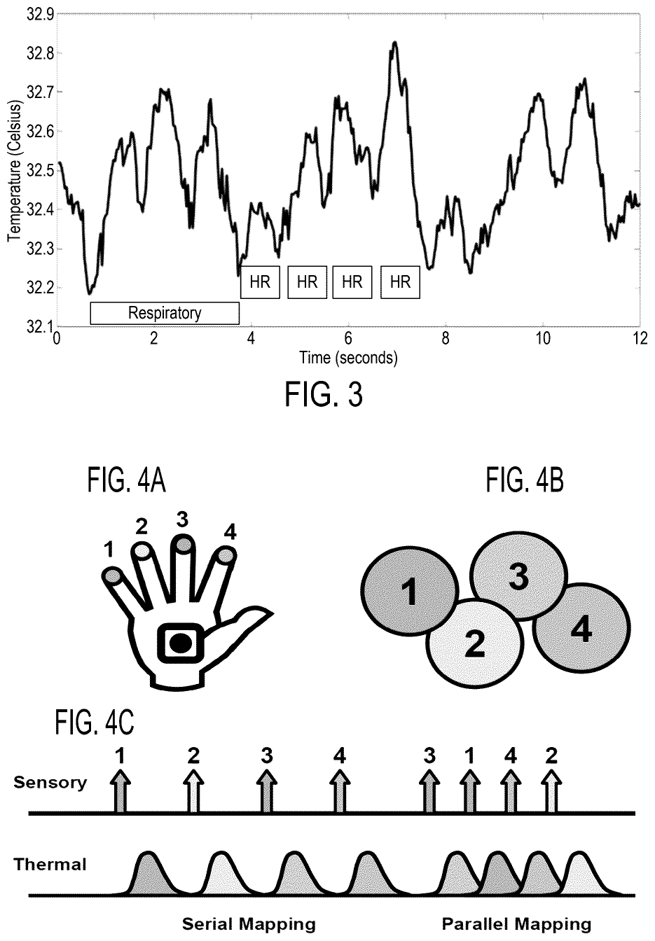

In one non-limiting example, motion correction can be implemented using an algorithm in which the thermal imaging data are cardiac gated by selecting images to process based on the temperature profile of a reference anatomical location (e.g., large pial artery). For instance, a pial artery time course is shown in FIG. 3. The cardiac variations (labeled as “HR”) and the respiratory variations (labeled as “Respiratory”) in the measured temperature can be readily identified due to the high quality, high resolution thermal imaging data. This reference location can be identified manually or automatically. As an example, the reference location can be identified in the image automatically via independent components analysis (“ICA”). This motion correction technique is advantageous in real-time mapping applications in order to allow increased processing speeds by effectively reducing the data sampling rate, as well as reducing the motion correction requirements by synchronizing with the cardiac pulsation.

To remove motion from patient movement and other sources, a rigid affine transformation can first be applied to each incoming frame. By utilizing a reduced portion of the data, the images can be processed in real time, such as to generate real-time functional maps during the performance of a task. Once the mapping is complete, a more rigorous method (e.g., non-rigid registration) can be applied to the data to generate the finalized map. The two step approach provides real-time feedback during mapping and then provides high quality data for the final mapping.

In another non-limiting example, motion correction can be implemented using an algorithm that leverages properties of brain (or other organ) motion to improve performance and decrease computation time. First, the brain's motion is a result of cardiovascular and respiratory cycles, and is therefore approximately periodic. As a result, motion does not need to be independently estimated for each frame. Instead the motion may be precisely calculated for several cycles, then extrapolated across the rest of the data. Additionally, the brain velocity function is smooth across space and time allowing it to be modeled by a spline function, which has far fewer parameters. This directly decreases both processing and memory requirements. Further, the brain motion is small between rapidly collected frames (e.g., every 3.3 ms), severely limiting the distance an individual pixel can travel between frames. This in turn limits the search space of deformations and decreases computational time. It also implies that the deformation function may be estimated by gradient-based optimization methods. Therefore, the motion may be estimated directly by minimizing the pixel-wise sum of squared differences between adjacent frames. As opposed to previous methods for correction of brain motion in intraoperative thermography, this approach is computationally efficient and does not result in blurring.

In one example implementation, motion correction begins with estimating rigid or nonrigid transformations between adjacent frames. The transformation can be defined as a 2D vector field over the image, where each pixel has a vector that describes its motion between the frames (e.g., a displacement field). Once this vector field has been found, the transformed image can be obtained by linear interpolation, by warping the source image according to the inverse of the displacement field, or so on. The first frame of the thermal video can be selected as a reference frame, and the displacement field can be calculated for all subsequent frames. In these instances, the displacement field is the field that minimizes the difference between the source and reference image. The algorithm can use a Newtonian optimization solver to quickly calculate displacement images that have been subsampled to meet computational resources.

Alternatively, the vector field can be directly estimated by optimizing the sum of squared differences between the transformed image and the target image. This method is effective, but may be too computationally expensive on large images. Alternatively, the transformation vector field can be downsampled and then the vector for each pixel can be linearly interpolated from the sampled pixels.

Because the motion of the brain surface is smooth over space (a pixel's vector is nearly identical to the vectors of neighboring pixels), subsampling the vector field dramatically decreases computational requirements with only a small loss in performance. Also because the magnitude of the motion is small across each frame (sampling at 30 frames per second), the linearly interpolated pixels can be approximated as a linear equation of downsampled pixels. This enables calculation of a simple, closed-form expression for the gradient for the vector optimization problem, using the sum of squared difference cost function.

In some implementations, a constrained solver can be used to efficiently obtain the solution. In this case, the physical constraints of deformation can be expressed as an inequality constraint in the vector field. This significantly limits the search space to a small hypercube about the origin. Furthermore, this optimization is approximately convex in this limited search space, so it is contemplated that the gradient descent will converge to an estimate of the deformation field that closely resembles the actual one.

Once the deformation field has been estimated between images, the field as a function of time is calculated. As the cardiac and respiratory rates are known, the deformation field between images can be calculated until a periodic function with approximately the same carrier frequencies as the cardiorespiratory rates is found and verified (e.g., over at least two periods).

The stability of the vector field can be tested over time to ensure the smoothness criteria described above are met. If outlier frames are detected, the deformation field can be interpolated from the fields of adjacent image pairs. Then, the periodic nature of the signal is leveraged, so that the deformation field can be estimated without direct computation for an arbitrary field as long as the frequencies remain stable.

Although the cardiac and respiratory rates are typically maintained nearly constant by an anesthesiologist, small deviations can be expected. In these instances, deformation fields can be estimated over the course of data collection and assessed for frequency drifts. The deformation function can be stretched or shrunk to fit the new frequency in the case of a deviation. Additionally or alternatively, the motion correction algorithms described in the present disclosure can be validated or otherwise augmented using visual-spectrum images collected with a video camera. Visual spectrum images contain more consistent features as the color of an object is typically stable over time.

The ITI systems described in the present disclosure provide an advantageous intraoperative testing environment that can automatically perform task administration and monitoring, and which can incorporate this information into the generation of functional maps in real-time. This environment is able to deliver hand motor, hand sensory, language, and cognitive tasks, among others. For tasks involving hand motor or sensory testing, patients will wear a wireless (or wired) haptic glove, which contains sensors that can track joint movement and tactile actuators on each finger that may deliver varying intensity of vibratory stimulation. Patients can receive task cues from the computer, either as a vibratory stimulus through the haptic glove (sensory), as an auditory cue from a speaker connected to the computer (motor/language/cognitive), or as a visual cue from a tablet computer set up in front of the patient (language/cognitive). Some tasks may require the patient to respond to the stimulus, and these responses can be measured through the haptic glove (motor tasks) or through a microphone array connected to the speaker (language/cognitive tasks). All behavioral data will be sent to the computer for storage and processing.

Such an automated approach has several advantages over manually administered mapping protocols. First, current task protocols have subjective assessment, and the results are therefore dependent on the surgical team. The choice of task and precise administration also varies across surgeons. These issues make it challenging to evaluate and optimize intraoperative testing across patients and surgical centers. Improvements to a manual task protocol identified at one center will likely not generalize as well to all glioma patients as compared to an automated task. Second, computers are capable of simultaneously administering multiple stimuli in parallel with high temporal precision. This enables the design and administration of highly efficient ITI mapping protocols. Further, glioma patients are typically older, and may have sensory disabilities from visual, hearing, or sensory losses. The computer stimuli can be easily modified to provide cues in a reliable communication medium for each patient, which will improve performance for disabled patients.

These and other benefits of automated tasks are magnified with ITI, as DES already has limitations for parallel network mapping. Automating tasks is possible with the ITI systems described in the present disclosure, which provide additional monitoring technology to ensure patient compliance and robust mapping.

As noted above, it is another aspect of the present disclosure to provide systems and methods for characterizing the spatial and temporal properties of the thermodynamic response, which can be used to optimize an infrared mapping procedure.

The thermodynamic response function (“TRF”) can be defined as the unit thermal response for functional activation, which represents the pattern of temperature change in time and space that occur after a focused stimulus. In this way, the TRF is the thermography analog of the fMRI hemodynamic response function (“HRF”). It is estimated that the TRF peak width is around 8-10 seconds, which is comparable to the BOLD HRF. Using the systems and methods described in the present disclosure, the impulse response function can be directly measured by measuring the brain temperature for a period of time (e.g., 30 seconds) after a brief stimulus (a long trial event design), which allows the brain to return to baseline before the next stimulus. Once the TRF has been determined, its spatiotemporal properties can be leveraged to map multiple functions in the same task (efficient multi-task mapping).

As shown in FIGS. 4A-4C, parallel mapping can be used to leverage organization of functional areas to improve efficiency. FIG. 4A shows an example of a haptic glove with four labelled stimulations. FIG. 4B shows an example spatial organization of the finger areas on sensory cortex. FIG. 4C shows an example of thermal responses to a sequence of stimuli. Mapping one finger at a time (serial mapping) is slower than interleaving stimuli when TRF overlap is small.

After preprocessing the thermal imaging data, the TRF for each subject can be estimated, such as by spatial independent component analysis (“ICA”). In some embodiments, a real-time implementation of ICA can be used. In general, spatial ICA decomposes the sequence of thermal images into a linear combination of components. Each component represents a set of pixels with coherent activity, and whose activity is statistically independent from the activity of all other components. The spatial ICA approach improves statistical power over pixel-based approaches by overcoming the multiple comparisons problem. The distribution of activity values for a component between task and baseline epochs (e.g., Kolmogorov-Smirnov test) can be used to find task-related components. The TRF for an impulse task (i.e., brief, focused stimulus) is then the sum of components with task-related activity. After measuring the TRF for each impulse task for each patient using both methods, signal properties (e.g., latency, duration, amplitude, spatial spread, temporal spread) can be computed. In some implementations, these signal properties can be compared to BOLD HRF from pre-surgical imaging data.

Individual TRF estimates from across patients can be aggregated to find a generalized TRF. While the shape of the TRF in space may vary significantly between tasks and individuals, the shape of the TRF over time is mostly conserved. Temporal ICA decomposes a set of time series into underlying source signals. Similar to the spatial ICA approach used above, the source signals are statistically independent. Therefore, the source signals will converge to aggregate TRF signals. This is similar to averaging across TRF signals, for the case where there are multiple distinct TRF subtypes. From the temporal ICA coefficients, TRFs from across subjects can be clustered, from which the variability in signal shape and duration can be estimated. The variability in spatial spread and TRF duration will influence the sensitivity and efficiency of network mapping.

Distinct impulse responses can be identified when their interaction effects are small (e.g., TRFs are far enough away from each other in space and/or time). As the impulse responses are brought closer together, the interaction effects grow and the spatial/temporal sensitivity is reduced or otherwise lost. It may then be desirable to estimate the smallest delay between two sensory stimuli where the resulting TRFs can still be separated. For example, each patient can perform a TRF estimation task using sensory stimuli due to the exact control of the timing, but the spacing between stimuli can be variable. The exact number of blocks and epoch timing can be determined from the TRF variability described above. The temporal spacing required to separate the two TRF functions can be estimated using the methods described to generate the TRFs.

The spatial spread of the TRF can also be estimated. In these instances, the patient can undergo a series of paired epochs, which contain two simultaneous tasks of the same sensory stimulation, but with differing location (e.g., thumb versus index finger). The spatial ICA analysis (described above) can be performed on the single and paired task portions together. The single task portion ensures that TRFs will emerge as ICA components, but including the paired task portion deconstructs these data in terms of the patient's own TRFs. The TRF amplitudes can be mapped to their corresponding stimuli, and the component magnitudes for each single task trial can be recorded.

Next, the paired task data can be analyzed. If the two tasks are separable, then the epoch will appear as a superposition of the two corresponding TRFs. This can be formalized by measuring the amplitude of both task components during their paired epoch. By comparing their values to the distribution of single-task TRF amplitudes, a probability that a TRF was observed can be calculated. The probability that both TRFs were independently observed is the product of both of their probabilities. It can be determined if two TRFs are separable used a probability threshold (value discussed below). If not, the task pair is repeated with a different stimulation pair (e.g., thumb versus middle finger) until the two TRFs are separable.

The optimal ITI task is directly related to the delay times needed to separate each impulse pair and the spatial separation required. It is desirable to maximize the total number of stimulation impulses delivered per unit time given constraints of the TRF.

Initial estimates of sensitivity and specificity versus DES can be determined from the patient data and used to develop a protocol. This allows for the optimization of the probability threshold for impulse separability to maximize concordance with DES. Whenever an impulse pair fails separability, the pair delay time is effectively sampled for a higher probability threshold. By starting with a conservative threshold (e.g., p=0.001), a large range of higher thresholds is intrinsically measured. Task results can be retroactively calculated for any observed probability by excluding any repeated epochs with lower probability. The probability that creates maps with the highest correspondence to DES can be picked. Further, these methods calculate the optimal task for only a single patient. By performing this protocol and analysis across many patients, a probability threshold and set of delay times that are compatible for all patients, or a group of patients, can be picked. This yields a final efficient multi-task mapping protocol that is both robust and highly efficient.

One advantage of ITI over DES is its potential to map functional networks. The ICA-based analysis techniques described above can be used to identify sets of brain regions that have related thermal activity. ICA is advantageous for network mapping because of its sensitivity to signal delays. Nodes of a network that activate with even small delays will naturally segregate into separate components.

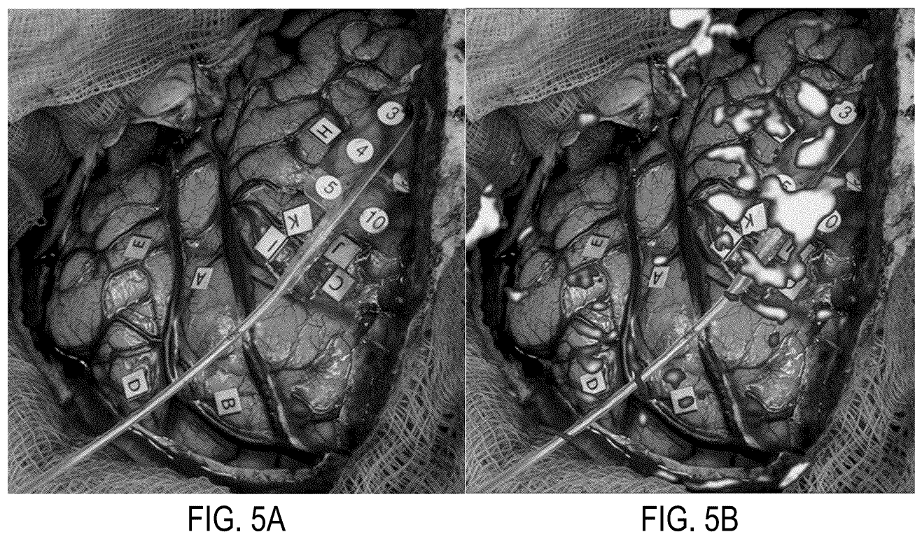

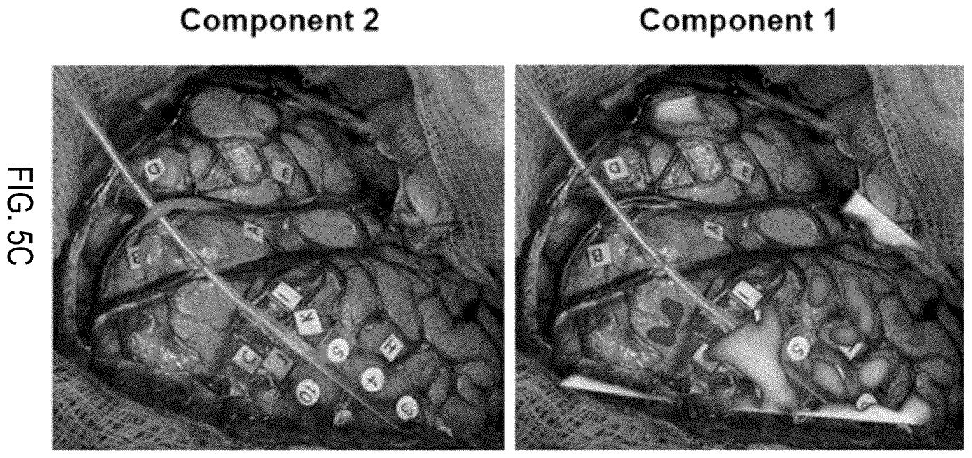

FIGS. 5A-5C illustrate this point. A visual-spectrum craniotomy image is shown in FIG. 5A and an ITI functional heat map overlaid on grayscale version of the same image is shown in FIG. 5B. Letters and numbers correspond to positive DES stimulation sites and electrocorticography grids, respectively. The same Picture Naming task was used for DES and ITI. ITI functional areas were determined with 1 minute of data, whereas, the DES mapping took 15 minutes. Data-driven networks were resolved using PCA from the same ITI data during the Picture Naming Task, as shown in FIG. 5C. Activity heat maps representing the first (FIG. 5C, top) and second (FIG. 5C, bottom) principal components are shown. The components have temporal differences during the Picture Naming task. Additional components would fill-in the activations shown in FIG. 5B.

To recover networks from ICA components, pairwise cross-correlation can be performed between the time series of all components. The peak correlation value represents the likelihood the two components are connected, and the position of the peak represents the delay between the components indicating causality. Hierarchical clustering, or other clustering algorithms, can be applied across all the pairwise component comparisons to identify clusters of components (basic network structure).

After a map of the network has been generated or otherwise obtained, its function can be further examined and/or analyzed. Brain areas can broadly be divided into excitatory (promoting a behavior), inhibitory (halting a behavior), or regulatory regions (modulating a behavior). DES tasks focus on excitatory and inhibitory regions, as its binary task design is not adapted for modulation areas. This may contribute to the lack of DES sensitivity in regulatory regions such as the supplementary motor area. However, these areas contribute significantly to function and are likely to be found in ITI. In some implementations, graph-theory techniques can be applied to elucidate interactions between nodes in the network. In this way, ITI-based node graph properties can be evaluated to determine if they are predictive of regions of disagreement with DES and related postoperative deficits.

Tumors are known to alter local hemodynamics through neovascularization. It is an aspect of the present disclosure that ITI can be used to monitor these changes. In some implementations, prior to ITI mapping, a patient can perform a series of short (e.g., 10-15s) breath holds. This induces a mild hypercapnic state, which causes vessels throughout the brain to dilate, increasing cerebral blood flow. The increased flow of warm blood from the body core will increase the overall surface temperature, and prominently highlight the major vasculature. However, tumor-induced vasculature lacks the autonomic control of healthy vessels, and will not dilate. Comparing the breath hold temperatures to the resting temperatures will enable measurement of local vasoreactivity, which can be used to infer areas near the tumor with heavy neovascularization. This physiologic vascular information may further improve the identification of resection boundaries.

It is another aspect of the present disclosure that ITI functional mapping can be used to help avoid postoperative neurologic deficits. It is contemplated that ITI-based functional mapping can be used to identify regions associated with neurologic defects that may otherwise not be measurable using DES alone. For example, if patients with new deficits have lower ITI specificity (mismatch with DES, i.e., ITI positive; DES negative), it may imply that ITI may be finding some functional areas that are missed by DES.

In addition to functional mapping, thermography is sensitive to “static” physiologic and pathologic factors. For instance, tissue with high tumor burden may be lower in temperature than the surrounding tissue due to a lower density of vessels or necrotic core or higher in temperature in a more vascularized lesion. These patterns can be leveraged as a method of identifying tumor margins. Furthermore, the thermal pattern can depend on perilesional and histologic features, which creates an opportunity for intraoperative classification of brain tumors using ITI.

Tumor grade is not typically known during surgery, so having a reliable estimate of tumor grade may influence how aggressive the resection is and reduce the need for reoperation. The class of a glioma is one of the most important factors in prognosis, and more aggressive gliomas may justify more aggressive resections. Because tumor progression is intricately tied to modification of local vasculature, and ITI is sensitive to local perfusion, it is contemplated that tumor class can be predicted from intraoperative thermal images. To provide more information about the vascular physiology, a vascular challenge (short breath-holding) that induces a mild hypercapnic state and globally increases cerebral blood flow and tissue perfusion can be used to enhance the vascular information to improve classification.

In some implementations, a machine learning algorithm, such as a convolution neural network, can be designed and trained to predict genomic tumor properties using baseline and breath-hold thermal images with presurgical MRI data. Pathologic and genomic data can be obtained from pathology reports and samples analyzed through the brain tumor institute. CNNs have a number of properties which make them advantageous for intraoperative glioma classification and treatment prediction, but other suitable artificial neural networks or machine learning algorithms can also be implemented.

As described above, the ITI system described in the present disclosure enables quantitative mapping that can be used for establishing precise surgical boundaries. Advantageously, the ITI system can implement one or more algorithms to convert these functional maps and tumor data into surgical variables. In this way, the ITI system can be programmed or otherwise configured to input functional maps and/or tumor data, generating output as surgical variable data, which may include control instructions for a robot-assisted surgical system, coordinate data for a surgical navigation or guidance system, or combinations thereof.

For instance, as described above, the ITI system can be used to map tumor extent. Lower-grade tumors generally exhibit significantly higher temperature than surrounding tissue due to heavy vascularization. However, high-grade tumors metabolically outpace their blood supply, creating a cold necrotic core. These physiological differences enable ITI to estimate tumor grade at the surgical bedside, potentially in combination with preoperative imaging. This information is not currently available until the surgery is complete, although it is critical in determining the resection boundary during the surgical procedure. The propensity for neovascularization also allows estimation of tumor burden, as thermal fluctuations are driven by metabolic and local hemodynamic properties. Combining these properties of thermal imaging with the functional mapping techniques described above, ITI can be developed into a comprehensive intraoperative imaging technique.

The systems and methods described in the present disclosure enable real-time image analysis of high-resolution, high-sensitivity thermal data. By concurrently estimating boundaries of both eloquent regions and the tumor itself, the precise optimal boundary of surgical resection can be calculated. In some implementations, mathematical formulas for boundary estimation can be tuned to incorporate preoperative imaging, predicted tumor grade, and individual patient expectations for quality and quantity of life. Integrating resection boundaries into the robot-assisted surgery system, surgical neuronavigation system, or the like, will allow for guiding the surgeon through completing a tumor resection that matches the individual needs of the patient. ITI-based resection may increase survival by permitting patient-specific aggressive resections with significantly reduced, or otherwise eliminated, postoperative neurologic deficits.

Methods for determining the probability that a pixel of tissue is functionally involved in a task have been previously described. By performing a series of motor, sensory, cognitive, and language tasks, a cumulative map of a patient's vital functions can be created. This cumulative map is an image of the exposed cortex, where each pixel has a probability of being functionally active. Similarly, the probability of tumor grade and tumor extent maps can also be produced based on imaging of static thermal parameters and preoperative imaging.

In many instances, patients undergo postoperative imaging (e.g., postoperative MRI), which can be used to identify the extent of resection. The extent of resection can be compared to preoperative imaging (e.g., preoperative functional MRI), in order to identify the proximity of the resection to the mapped functional areas. These can be cross-referenced with the ITI-based functional maps, such as by using a surface-to-volume co-registration algorithm. The probability of each pixel being either tumor or functionally active can be compared between pixels that were resected and pixels that were not.

In some implementations, a machine learning algorithm (e.g., a support vector machine or other suitable machine learning algorithm) can be trained to distinguish resected pixels from non-resected pixels based on these two probability values, as well as tumor grade and other patient demographic variables. This trained machine learning algorithm can then be used to identify precise resection boundaries, which can be expressed mathematically as a volume in three-dimensional space. In this way, following ITI-based functional mapping, the surgical team can receive not only a functional map of the brain, but also a recommended surgical resection that balances maximal removal of tumor tissue with protecting functional areas.

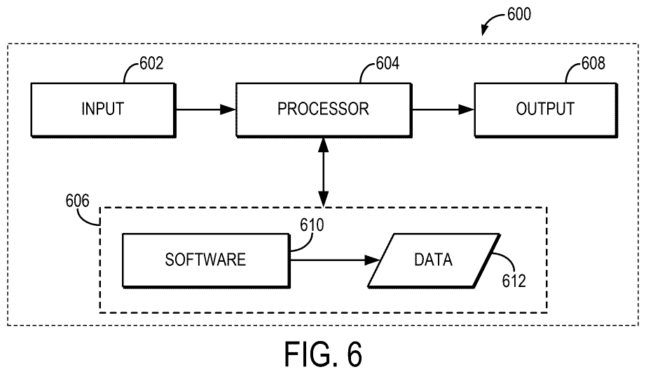

Referring now to FIG. 6, a block diagram of an example of a computer system 600 that can perform the methods described in the present disclosure is shown. The computer system 600 generally includes an input 602, at least one hardware processor 604, a memory 606, and an output 608. Thus, the computer system 600 is generally implemented with a hardware processor 604 and a memory 606.

In some embodiments, the computer system 600 can be a workstation, a notebook computer, a tablet device, a mobile device, a multimedia device, a network server, a mainframe, one or more controllers, one or more microcontrollers, or any other general-purpose or application-specific computing device.

The computer system 600 may operate autonomously or semi-autonomously, or may read executable software instructions from the memory 606 or a computer-readable medium (e.g., a hard drive, a CD-ROM, flash memory), or may receive instructions via the input 602 from a user, or any another source logically connected to a computer or device, such as another networked computer or server. Thus, in some embodiments, the computer system 600 can also include any suitable device for reading computer-readable storage media.

In general, the computer system 600 is programmed or otherwise configured to implement the methods and algorithms described in the present disclosure. For instance, the computer system 600 can be programmed to control a thermal camera to acquire thermal imaging data, to deliver stimuli to a patient, to process thermal imaging data to generate functional maps, and to otherwise control the operation of an ITI system.

The input 602 may take any suitable shape or form, as desired, for operation of the computer system 600, including the ability for selecting, entering, or otherwise specifying parameters consistent with performing tasks, processing data, or operating the computer system 600. In some aspects, the input 602 may be configured to receive data, such as data acquired with a thermal imaging camera and/or a visual spectrum camera, in addition to behavioral data acquired using one or more input peripheral devices (e.g., a haptic glove, a microphone or microphone array). Such data may be processed as described above to generate functional maps indicative of neuronal activity. In addition, the input 602 may also be configured to receive any other data or information considered useful for implementing the methods described above.

Among the processing tasks for operating the computer system 600, the one or more hardware processors 604 may also be configured to carry out any number of post-processing steps on data received by way of the input 602.

The memory 606 may contain software 610 and data 612, such as data acquired with a thermal imaging camera, a visual spectrum camera, or one or more input peripheral devices, and may be configured for storage and retrieval of processed information, instructions, and data to be processed by the one or more hardware processors 604. In some aspects, the software 610 may contain instructions directed to controlling a thermal camera to acquire thermal imaging data, delivering stimuli to a patient, processing thermal imaging data to generate functional maps, or otherwise controlling the operation of an ITI system.

In addition, the output 608 may take any shape or form, as desired, and may be configured for displaying visual stimuli to a patient, generating audio stimuli, providing vibratory stimuli via a haptic glove, displaying thermal images, displaying functional maps, in addition to other desired information.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. An intraoperative thermal imaging system, comprising:
a thermal camera;
one or more peripheral devices; and
a computer system comprising a processor and a memory, the computer system being configured to:
receive thermal imaging data from the thermal camera;
receive behavioral data from the one or more peripheral devices; and
generate a functional map indicative of neuronal activity in a subject using the thermal imaging data and the behavioral data.

2. The intraoperative thermal imaging system as recited in claim 1, wherein the one or more peripheral devices comprise at least one of a monitor, a speaker, a microphone, or a haptic device.

3. The intraoperative thermal imaging system as recited in claim 2, wherein the haptic device is a haptic glove.

4. The intraoperative thermal imaging system as recited in claim 3, wherein the computer system is configured to receive behavioral data from the haptic glove and compute therefrom a motion trajectory of the haptic glove.

5. The intraoperative thermal imaging system as recited in claim 4, wherein the computer system is configured to perform quality assurance on functional task performance of a subject wearing the haptic glove.

6. The intraoperative thermal imaging system as recited in claim 2, wherein the microphone comprises a microphone array.

7. The intraoperative thermal imaging system as recited in claim 6, wherein the computer system is configured to receive audio data recorded by the microphone array and to isolate speech from a subject in the audio data.

8. The intraoperative thermal imaging system as recited in claim 7, wherein the computer system is configured to convert the isolated speech to text data and to compare the text data to a list of expected responses corresponding to a functional task.

9. The intraoperative thermal imaging system as recited in claim 1, further comprising a base unit comprising a mobile cart, wherein the thermal camera and the one or more peripheral devices are coupled to the base unit.

10. The intraoperative thermal imaging system as recited in claim 9, wherein the thermal camera is coupled to the base unit via a moveable support coupled on one end to the base unit and on its other end to the thermal camera.

11. The intraoperative thermal imaging system as recited in claim 9, wherein the computer system is housed within the base unit.

12. The intraoperative thermal imaging system as recited in claim 1, wherein the computer system is configured to generate and provide task cues to a user, the task cues defining a functional task for the user to perform.

13. The intraoperative thermal imaging system as recited in claim 12, wherein the one or more peripheral devices comprise a haptic glove and the task cues comprise a vibratory stimulus generated by the haptic glove.

14. The intraoperative thermal imaging system as recited in claim 12, wherein the one or more peripheral devices comprise a speaker and the task cues comprise an auditory cue.

15. The intraoperative thermal imaging system as recited in claim 12, wherein the one or more peripheral devices comprise a display and the task cues comprise a visual cue.
